(19) United States
(12) Patent Application Publication
(10) Pub. No.: US 2007/0099225 A1
(43) Pub. Date: May 3, 2007

(54) Separation of chromosomes using an affinity-based magnetic bead separation in suspension

(76) Inventors: George S. Wilson, Lawrence, KS (US); Samadhi Nipunika Vitharana, Fremont, CA (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/592,456

(22) Filed: Nov. 3, 2006

Related U.S. Application Data
(60) Provisional application No. 60/733,155, filed on Nov. 3, 2005.

(51) Int. Cl. C12Q 1/68; C07H 21/04; C12N 1/08

(52) U.S. Cl. 435/6; 435/270; 536/25.32

(57) ABSTRACT

A method for separating and isolating a target chromosome from a cellular sample by using a DNA probe with a fluorescein tag. Magnetic beads bound to anti-fluorescein isothiocyanate antibody were reacted with the fluorescently labeled pool of chromosomes and then separated in suspension by exposure to a magnetic field.

### CROSS-REFERENCE TO RELATED APPLICATIONS

This application is based on and claims priority to U.S. Provisional Application Ser. No. 60/733,155, filed on Nov. 3, 2006, which is incorporated herein by reference.

### STATEMENT REGARDING FEDERALLY SPONSORED RESEARCH OR DEVELOPMENT

Not applicable.

### BACKGROUND OF THE INVENTION

Chromatin structure is believed to play a pivotal role in regulating genes, in addition to its function in packaging the DNA into the nucleus. The interest and effort previously devoted to exploring chromatin structure and its influence on genetic control have increased dramatically, with the rapid evolution of proteomics research after the deciphering of the human genome. Even though a single eukaryotic cell may contain thousands of genes, only those that are required for the function of a particular cell are expressed. The others are repressed by some regulatory mechanism. Another aspect of regulation is the underlying mechanism of molecular imprints, which are inheritable markers that indicate the transcriptional state of a gene based on parental origin. Although there is ample evidence to support the fact that chromatin is an integral part of such regulation of gene expression, the details at the molecular level remain poorly understood. A map of chromatin structure, therefore, in defined genomic intervals across the human genome will be a useful tool to understand the impact of such epigenetic processes on the control of genes. What is not yet well understood is what modifications of histones, either as variants in the amino acid sequence either as variants in the amino acid sequence (S. B. Hake and C. D. Allis, Proc. Natl. Acad. Sci. U.S., 2006, 103: 6428-6435) or through histone post-translational modifications (“PTMs”) (T. Jenuwein and C. D. Allis, Science, 2001, 293: 1074-1080), influence the readout, replication and repair of DNA. Histone proteins are shown to be post-translationally modified at both the N-terminal and C-terminal ends, as well as in their interiors. It could either be as simple as loosening the grip of histones on DNA upon acetylation of histones, allowing transcription machinery to access the DNA template in chromosomes, or a more complicated combinatorial process involving different types of modifications acting synergistically or sequentially, to mark a gene as transcribable or not. Acetylation and de-acetylation have already been demonstrated as effectors of transcription, but the more detailed picture may prove to be much more complicated. Such modifications are known to rapid and dynamic, with half-lives in the order of minutes (J. H. Waterborg, Biochem. and Cell Biol. 2002, 80: 363-378). A quantitative analysis of the different modifications of histones in a region of interest in a particular chromosome is, therefore, the ultimate picture one would desire to see. A map that illustrates changes of histones, residue by residue, will enable a comparison of the modifications in a patient cell line with that of a control and determine if the gene regulation is influenced by such modifications in genetic diseases.

A multitude of techniques have been used to study the different PTMs of histones. Although tools such as immunofluorescence will provide a qualitative picture of the protein modifications and the genetic information, respectively, such techniques lack the resolution required to understand the underlying biochemistry of chromatin. A more recently discovered technique, which is widely used currently to study PTMs, especially acetylation, is the chromatin immunoprecipitation assay (ChiP). This is an indirect way to study histones, because it is based on the analysis of DNA that has reacted with the histones. While this technique is an advancement in the analysis of PTMs of histones, the specificity of the method, however, relies entirely on the quality of the antibodies used. Potential cross-reactivity of antibodies may result in co-precipitation, if the antibodies are not characterized thoroughly against all the possible modifications. It is further quite unlikely that PTMs including methylation, acetylation, and phosphorylation can be simultaneously tracked in combinatorial fashion. Another limitation is the difficulty in assessing the absolute levels of the different types of histone modifications in a cell simultaneously. This has led the chromatin research community in the direction of mass spectrometry (“MS”) to unravel the chromatin structure on a residue-by-residue basis. Although it is not debatable that MS will be capable of providing more details than any other technique available and used so far to study chromatin, current MS work performed in chromatin research generally involves the analysis of only bulk chromatin. In other words, it is the histones extracted from the entire genome that have been analyzed mass spectrometrically to study PTMs. Although this approach may give insight as to what positions and residues can be post-translationally modified at a specific functional group, it provides little evidence about the specific modifications in a particular genomic region of interest. The latter is of far more importance, as it can shed light on what is happening genetically and epigenetically in a region of the genome that leads to a disease. The challenge, therefore, lies in the isolation and analysis of chromatin structure associated with specific genes. In the context, for example, of Prader-Willi syndrome (“PWS”), a pediatric genetic disease, the responsible genes are all clustered in a region 4 Mb in length on the long or q arm of chromosome 15. To compare the PTMs of histones associated with PWS genes in affected and normal individuals, histones need to be extracted only from the region of interest. The specific segment, which could be a single gene, a promoter region, or multiple genes in the PWS region, needs to be first clipped from the rest of the target chromosome to extract histones from that region. The first step in the sample preparation process for this methodology that is yet to be developed is enrichment of the target chromosome, e.g. chromosome 15. Having a sufficient amount of histones for MS analysis is a significant consideration.

The main objective of the present invention is to overcome the major shortcomings associated with sorting chromosomes by flow cytometry, the current method for fractionating chromosomes. The time required to obtain sufficient material for MS analysis by flow sorting of chromosomes is based on the assumption that 1 pmol of histone H4 would need to be analyzed in a gene of 5000 bp. This would require about 10¹⁰ chromosomes containing the gene of interest. Assuming a flow cytometer sorting speed of 10³ per second at 90% efficiency, preparing sufficient material would require 2500 hours (15 weeks) of continuous operation, a clearly impractical approach.

The availability of chromosome-specific, repetitive sequence hybridization probes presents the possibility of a unique alternative method for chromosome isolation. The approach involves the combination of in-situ hybridization of such labeled probes to chromosome mixtures, followed by isolation based on the presence of the hybridized probe using magnetic beads. However, to date, there have only been a handful of attempts to develop such a technique. For example, in Dudin, et al., Sorting of chromosomes by magnetic separation, Hum. Genet. 80 (1988) 111-116, chromosomes from Chinese hamster hybrid cells containing four and nine human chromosomes was used. In-situ hybridization using a biotin-labeled probe was carried out on suspensions of chromosomes. Streptavidin was covalently coupled to the surface of magnetic beads, and these were incubated with the hybridized chromosomes. However, others have found that the Dudin approach was not very successful due to problems with adventitiously adsorbed contaminants, chromosome aggregation, and losses during centrifugation steps. In a separate experiment, Kausch and Narayaswami also reported the sorting of a human chromosome from a mouse-human hybrid cell line using a biotin-labeled DNA probe. The chromosomes were immobilized on a solid support using a cross-linker, and the biotin-labeled DNA probe was then used to identify the target chromosome using streptavidin-linked magnetic particles. See Kausch et al., U.S. Pat. No. 5,508,164. Again, there were difficulties removing the chromosome from the magnetic bead when using the biotin-streptavidin system. Moreover, clumping of the chromosomes remains a problem when immobilization techniques are used. Moreover, in both the Dudin and Kausch approaches, hybridization strategy was used to isolate chromosomes in somatic hybrids such as human-hamster cell lines, in which a single human chromosome is inserted into a hamster cell line. This insertion and subsequent growth in a different cell line may cause major changes in the regulatory functions of human chromatin. Thus, this is not an appropriate method to pursue if the objective is to investigate the native state of human chromatin.

### BRIEF SUMMARY OF THE INVENTION

The present invention is directed to method for separating and isolating a target chromosome using an affinity-based separation in which the target chromosome, recognized by a specific probe, is separated using magnetic particles.

In the present invention, the chromosomes are sorted in a massively parallel way, as opposed to sorting in series by flow cytometry. Consequently, the time required to get a large quantity of material for any type of analysis is minimal because the isolation time does not depend on the number of cells that need to be sorted. Within about 3 to 4 days, the complete procedure developed in the present invention can be performed and a separated pool of the target chromosome can be obtained. It will be appreciated that this time may be further decreased by automating various steps in the process.

In another aspect, by the use of a specific probe to identify the target chromosome, the nonspecificity of the standard method that is based on the identification of chromosomes on nonspecific fluorescent, DNA binding or intercalating dyes may be eliminated or substantially reduced.

In the present invention, the magnetic bead approach takes advantage of the specificity of the centromere probe. The mitotic chromosomes extracted by blocking cultured cells at metaphase are then reacted with chromosome-specific centromere probes with a fluorescent label in suspension, to mark the target chromosome that will be separated.

In the present invention, a fluorescent probe is used. The fluorescent probe has at least two important advantages over biotin-labeled probes. First, the fluorescein label allows the investigator to spot the target chromosome in a chromosome spread. Second, the magnetic particle can be easily removed from the target chromosome after the separation. This is next to impossible when using the avidin-biotin link based on a biotin-labeled probe.

In another aspect, the fractionation process of the present invention occurs in a suspension containing the target chromosome. This reduces the clumping that occurs when the chromosomes are immobilized on substrate.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 shows metaphase chromosomes extracted from cells after about 12 to 13 hours colcemid treatment to arrest cells at metaphase. Chromosomes were extracted by rupturing the swollen cell membranes by applying pressure by vortexing. DNA was stained with 4′,6-diamidino-2-phenylindole dihydrochloride (“DAPI”) for identification (40× original magnification).

FIG. 2 shows the specificity of the chromosome 15 centromere probe (green signal) used for the fractionation process. The probe was reacted against a metaphase spread along with a chromosome 15 telomere probe (red signal). As the white arrows indicate, both the red and the green signals appear on the same two chromosomes on the spread, confirming the specificity of the chromosome 15 centromere probe.

FIG. 3 shows the control experiment for verification with the paint probe (40× original magnification). (A) An aliquot of chromosome suspension right after extraction from cells, prior to fractionation, subjected to FISH with chromosome 15 paint probe. The chromosomes are stained with DAPI for identification of the entire population of chromosomes. (B) The above chromosomes under FITC filter to detect the painted chromosomes (40× original magnification). *Painted/total ratio is 6%. (C and D) Characterization of the isolated pool of chromosomes with FITC-labeled chromosome 15 paint probe. (C) DAPI-stained fractionated pool of chromosomes (40× original magnification). (D) FITC-labeled chromosomes in the total population shown in (C).

### DETAILED DESCRIPTION OF PREFERRED EMBODIMENT

The present invention relates to a method for isolating and separating a target chromosome from a cellular sample. The method comprises the steps of (1) obtaining a cellular sample having a plurality of chromosomes, including the target chromosome; (2) arresting the cellular sample in metaphase; (3) extracting the plurality of chromosomes, including the target chromosome from the cellular sample; (4) labeling the target chromosome with a nucleic acid probe having a fluorescent reporter group to form a labeled target chromosome; (5) contacting the labeled target chromosome in suspension with an antibody against the fluorescent reporter group, with the antibody covalently linked via a linker to a magnetic bead; and (6) exposing the suspension to a magnetic field to separate the target chromosome from the plurality of chromosomes. In the present invention, the extraction step generally involves lysing the cellular sample with a hypotonic solution to form a suspension which contains the chromosomal material from the sample, as well as other non-chromosomal cellular debris. Non-chromosomal cellular debris is removed by centrifuging and washing with a chromosome isolation buffer. A polyamine chromosome isolation buffer comprising spermine and spermidine is preferably used. The separation process results in an enriched pool of the target chromosome, with 73% or more purity, and preferably 80% or more purity.

As used herein, the term “antibody” embraces polyclonal and monoclonal antibodies, chimeric antibodies, haptens and antibody fragments, and molecules which are antibody equivalents in that they specifically bind to an epitope on the antigen of interest. The term “antibody” includes polyclonal and monoclonal antibodies of any isotype (IgA, IgG, IgE, IgD, IgM), or an antigen-binding portion thereof, including, but not limited to, F(ab) and Fv fragments such as sc Fv, single chain antibodies, chimeric antibodies, and humanized antibodies.

As used herein, the term “cellular sample” embraces a single cell as well as a plurality or population of cells. The cellular sample is preferably a eukaryotic cell, and is most preferably from a mammal, such as dogs, cats, pigs, chimpazeees, monkeys, rodents including mice, rats, and hamsters, and humans. Most preferably, the chromosomes in the cellular sample are all human chromosomes.

As used herein, the term “chromosome” embraces a heredity-bearing gene carrier of a living cell which is derived from chromatin and which comprises DNA and protein components (especially histones). The conventional internationally recognized individual human genome chromosome numbering identification system is employed herein. The size of an individual chromosome can vary from one type to another with a given multi-chromosomal genome and from one genome to another. In the case of the (preferred) human genome, the entire DNA mass of a given chromosome is usually greater than about 100,000,000 bp. In a preferred aspect, the target chromosome is a human chromosome which is associated with a genetic disease. In one embodiment, the target chromosome is human chromosome 15.

As the term is used herein, “separated from” or “separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “separated from” a population of second substances has a measurably lower content of second substances as compared to the non-separated mixture of first and second substances.

The nucleic acid probes used in the present invention are most preferably complementary to the target DNA sequence associated with the target chromosome, i.e., there is a 100% complementary match between the probe nucleotides and the target sequence. More broadly, less than 100% correspondence probes can be used, so long as the probes adequately hybridize to the target sequence. In one aspect, there should be at least about 80% sequence identity between the probe and a sequence which is a complement to target sequences, more preferably at least about 90% sequence identity. Methods for calculating sequence identity are known to those skilled in the art.

The probes of the invention preferably should have a length of at least about 300 nucleotides, and more preferably at least about 1000 nucleotides. In general, the probe is about 300 to 1500 nucleotides in length, with ranges between 1000 to 1500 being most preferred.

The probes also preferably target a DNA sequence in the centromere of the target chromosome. The term “centromere” refers to a heterochromatic region of the eukaryotic chromosome which is the chromosomal site of attachment of the kinetochore. The centromere divides just before replicated chromosomes separate, and holds together the paired chromatids.

The fluorescent reporter group utilized in conjunction with the probes includes standard FISH dyes, such as fluorescein, rhodamine, Texas-Red and cascade blue, umbelliferone, dichlorotriazinylamine fluorescein, dansyl chloride, Cy-dyes, Alexa-dyes, phycoerythrin, and others known to those skilled in the art. The reporter group is preferably a fluorescein isothiocyanate. The term “fluorescent” (and equivalent terms) has general reference to the property of a substance (such as a fluorophore) to produce light while it is being acted upon by radiant energy, such as ultraviolet light or x-rays.

The magnetic particles of the present invention may be comprised of any suitable magnetic material. Typically, the particles comprise ferric oxide. The magnetic particles of the present invention are from about 2 nm to about 10 microns in diameter and preferably are from about 50 nm to about 2 microns in diameter.

Methods for linking the magnetic particle to the antibody against the fluorescent reporter group are also known in the art. As used herein, the term “linker” embraces a molecule that joins two other molecules together. The antibody is linked to the magnetic particle using a suitable linker homobifunctional or heterobifunctional linker. A representative list includes DTSSP, SPDP, SAED, SMPT, DPDPS, DSP, BSOCOES, sulfo-EGS, APDP, DTBP, BASED, or SADP, with the most preferred being DTSSP or SPDP. Of these, ethylene glycol bis(sulfosuccinimidylsuccinate (sulfo-EGS) is most preferred.

Linkage between the antibody and the magnetic particle may be reversed using a suitable reversing agent. For example, DTSSP is reversible by thiol reduction, BSOCOES is base cleavable, and EGS and sulfo-EGS are cleavable by hydroxylamine. Periodate cleavable linkers also may be used in the practice of the invention. Polymers such as alginate are reversible by calcium ion.

The following paragraphs illustrate the present invention using the separation and isolation of human chromosome 15 from a lymphoblast cell line as an exemplary embodiment.

Solution Materials and Methods

All of the following solutions were prepared and filtered through a 0.22-micron filter: 1×SSC (0.15 M sodium chloride, 0.015 M sodium citrate, pH 7.0); Ohnuki's hypotonic solution (5 ml 55 mM sodium nitrate, 2 ml 55 mM sodium acetate, 10 ml 55 mM potassium chloride); 100× Denhardt's solution (10 g Ficoll 400, 10 g polyvinylpyrrolidone MW 360,000 (Cat. No. PVP360; Sigma-Aldrich, St. Louis, Mo., USA), 10 g BSA fraction V (Cat. No. A7906; Sigma-Aldrich) in a total volume of 500 ml); and IB+M solution (50 mM potassium chloride, 5 mM Hepes, 10 mM magnesium sulfate, pH 8.0).

To prepare the polyamine chromosome isolation buffer, Stock A containing 150 mM Tris-HCl, 20 mM EDTA, 800 mM KCl, 200 mM NaCl in 100 ml water was prepared. Stock B contained 5 mM EGTA in 100 ml water. Then, 5 ml each of stock A and stock B were mixed with 40 ml of Nanopure water, pH 7.2, 50 μl of beta-mercaptoethanol (Cat. No. M-6250; Sigma Aldrich). To 25 ml of the above, 30 mg digitonin (Cat. No. D141; Sigma-Aldrich) was added and incubated for 40 minutes at 37° C. and then sterile filtered. Then, 12.5 μl each of 0.4 M spermine (Cat. No. S4264; Sigma-Aldrich) and 1.0 M spermidine (Cat. No. 85558; Fluka Biochemika, Sigma-Aldrich) were added.

Cell Culture

The lymphoblastoid cell line (GM01056C; NIGMS Cell Repository, Camden, N.J., USA) was cultured with 10% heat-inactivated fetal bovine serum (“FBS”) (Cat. No. 10082-147; GIBCO Invitrogen Corp., Carlsbad, Calif., USA), 1% L-glutamine (Cat. No. 25030-149; previously GIBCO BRL Life Technologies, currently GIBCO Invitrogen Corp.), and 1% penicillin-streptomycin (Cat. No. 15140-148; previously GIBCO BRL Life Technologies, currently GIBCO Invitrogen Corp.), in RPMI 1640 medium (Cat. No. 9161; Irvine Scientific, Santa Ana, Calif., USA). The cells were fed once every 2 to 3 days depending on the cell cycle.

Metaphase Arrest

The first step in the separation process involves arresting the cells of the cellular sample at metaphase. Chromosomes are in their most compact form in metaphase, the stage of the cell cycle at which individual chromosomes can be identified, which is important to isolation of a target chromosome.

Briefly, cells were blocked at metaphase by adding 0.1 μg of colcemid (Cat. No. 15210-040; GIBCO Invitrogen Corp.) per milliliter of lymphoblastoid cell culture for about 12 to 13 hours. Cells were pooled on ice and counted. Cells were aliquoted into 15-ml or 50-ml polypropylene centrifuge tubes aiming for a final concentration of 6 to 14×10⁶ mitotic cells/tube. The number of mitotic cells was calculated assuming the number was about 30% of the total cell count. The cells were centrifuged between 800 and 1000 rpm for 8 minutes at 2° C. The supernatant was aspirated and the cell pellet flicked.

By exposing the cell culture to colcemid, a majority of the cells were arrested at metaphase to increase the yield of mitotic chromosomes. It should be noted that upon extended exposure to colcemid, which was about 12 to 13 hours for lymphoblastoids, chromosomes become highly condensed and lose their native morphology, making them difficult to identify. Unlike cytogenetic analysis, which is heavily dependent on chromosomal morphology, this is not anticipated as a problem in the present invention.

Lysis and Extraction of Mitotic Chromosomes

The next step in the separation process involves cell lysis and extraction of the mitotic chromosomes. Several problems have been encountered during extraction and maintenance of mitotic chromosome preparations in the prior art. The main problem has been chromosome aggregation. This has been mitigated by using two previously reported buffers used in flow cytometry. First, a magnesium sulphate (MgSO₄) buffer was utilized in Van den Engh et al., Preparation of chromosome suspensions for flow cytometry, Cytometry 5 (1984) 625 108-117. Second, polyamine buffer was used in Cram et al., Polyamine buffer for bivariate human flow cytogenetic analysis and sorting, in: Z. Darzynkiewicz, H. A. Crissman (Eds.), Methods in Cell Biology, Flow Cytometry, Vol. 33, Academic Press, San Diego, 1990, pp. 377-383.

In the present invention, a modification of the method for extracting chromosomes using polyamine chromosome isolation buffer for flow cytogenetic analysis and sorting was preferably used. The modified polyamine method was used to preserve the total DNA content of the chromosomes, as opposed to the MgSO₄ method, which increases the activity of endogenous nucleases due to the presence of divalent cations. In the polyamine procedure, spermine, (CH₂)₄[(NH₂(CH₂)₃NH₃)₂]⁴⁺, and spermidine, [H₃N(CH₂)₃NH₂(CH₂)₄NH₃]³⁺, were added to the buffer, to replace the divalent cations in the MgSO₄ protocol to stabilize the integrity of the chromosomes. The presence of polyamines in the extracting buffer tends to extract proteins such as high-mobility-group proteins, TATA-binding proteins, and transcription factors, whereas the nucleosome array remains undisturbed. See Wallrath et al., Mapping chromatin structure in Drosophila, in: H. Gould (Ed.), The Practical Approach Series: Chromatin, a Practical Approach, Oxford Univ. Press, Oxford, 1998, 633 pp. 59-77. This assumption was supported by performing an immunofluorescence experiment with a primary anti-histone antibody for all acetylated isomers on intact chromosomes as well as extended chromatin fibers derived from chromosomes extracted in this manner (data not shown). The secondary antibodies used to identify the primary antibodies carry an FITC fluorescent tag. Therefore, the signals seen with FITC labels indicate the presence of histones, confirming their integrity after exposure to the polyamine procedure.

To rupture the cell membranes for the extraction process, the cells were exposed to a hypotonic solution, preferably Ohnuki's hypotonic for lymphoblastoid cell lines, which swells the cell membranes. However, immediately prior to addition of the hypotonic solution to swell the cells, 12.5 μl each of spermine and spermidine (both polyamines) was added to 25 μl of Ohnuki's hypotonic solution.

The amount of hypotonic solution used to enhance lysing of the membranes is dependent on the number of mitotic cells present in the culture. For example, for a culture containing mitotic cells ranging from 6 to 14×10⁶, the amount of hypotonic solution varied from about 2.5 to 5.5 ml. A total cell count was done and, as noted above, the mitotic fraction was assumed to be 30% of the total cells. The cells were allowed to swell for 70 to 90 minutes at room temperature and then centrifuged at 1000 rpm for about 5 minutes at room temperature. Supernatant was aspirated and the cell pellet flicked.

A volume of 1.0 ml of polyamine chromosome isolation buffer was added to each tube and then flicked. The swollen cells were then subjected to vortexing to break them open and release the chromosomes into the chromosome isolation buffer containing the polyamines. Each tube was vortexed vigorously for 15 seconds and then placed on ice for at least 5 minutes. An aliquot of the suspension was examined under fluorescence by staining with 4′,6-diamidino-2-phenylindole dihydrochloride (“DAPI”) (Cat. No. D9542; Sigma-Aldrich) or 3,8-diamino-5-(3)-diethylaminopropyl)-6-phenylphenanthridinium iodide (propidium iodide; “PI”) (Cat. No. P4170; Sigma-Aldrich), which are both DNA-intercalating dyes. Until mitotic chromosomes were leased by a majority of cells, the suspension was vortexed for about 45 to 90 seconds in total, in about 15-second increments. However, it is important to note that too much vortexing made the longer chromosomes appear stringy and, thus, was avoided.

Isolation of Chromosome from Other Cellular Debris

The extracted chromosomes coexist with the lysed membranes and the remaining organelles of the cells as cellular debris in suspension. Thus, the next step in the fractionation process involves isolation of the chromosomes from other cellular debris. To achieve this, a specific protocol developed by the Bio Sciences Division of the Los Alamos National Flow Cytometry Resource of Los Alamos National Laboratories to extract chromosomes of good morphology and remove most of the nuclei and other extraneous cellular debris was adopted. See Fawcett et al., Large-scale chromosome sorting, in: Z. Darzynkiewicz, J. P. Robinson, H. A. 636 Crissman (Eds.), Methods in Cell Biology, Academic Press, San Diego, 1994, pp. 319-330. This procedure involves several centrifugation steps followed by filtering through a nylon mesh with 62-μm pores. More specifically, extracted chromosomes were centrifuged at 50 g for three minutes and the supernatant containing the chromosomes was transferred to a separate centrifuge tube. This suspension was recentrifuged at 50 g for another three minutes and the supernatant removed into a fresh centrifuge tube to get rid of most of the nuclei and cellular debris present in the chromosome extract. To purify the chromosomes further, the suspension was drawn into a 1-cc or 3-cc syringe through an 18-gauge×1½-inch disposable needle and then passed through a sterilized nylon mesh wedged between the syringe and a fresh 18-gauge×1½-inch needle.

The concentration of the extracted chromosomes was determined by staining an aliquot (a known volume) with a DNA-binding dye such as DAPI, which was then observed under fluorescence to determine the number of chromosomes in that aliquot. One microliter of a chromosome suspension with about 10⁶ chromosomes/ml sample was diluted in a total of 1 ml of polyamine buffer and fixed by adding 20 drops of 3:1 methanol:acetic acid fixative while gently swirling the suspension. Chromosomes were then centrifuged at 350 g for 10 minutes and the supernatant was removed. A volume of 1 ml of the same fixative was added to the flicked pellet while vortexing the tube at the lowest setting and incubated at room temperature for 30 minutes. After centrifugation under the same conditions, the supernatant was removed and 1 ml of fresh fixative was added.

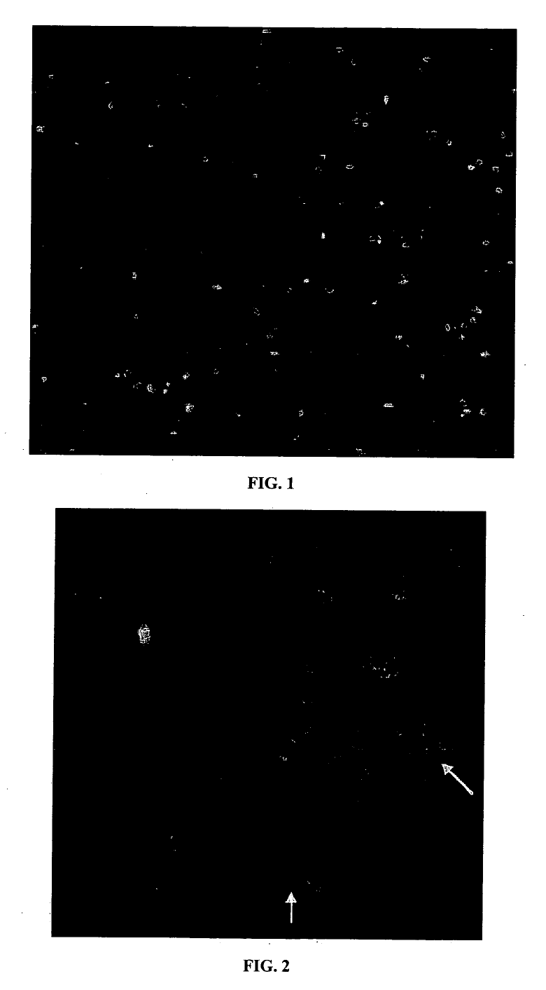

An image of some extracted chromosomes that were subjected to this entire purification protocol under optimal conditions is shown in FIG. 1.

Labeling Chromosome 15 by FISH in Suspension

The next step in the separation process involves labeling of the target chromosomes in suspension to allow for the eventual magnetic bead separation. After centrifugation and removal of the supernatant, the chromosome pellet was resuspended in 160 μl of prewarmed hybridization buffer (40% deionized formamide, 4×SSC, 2× Denhardt's solution). See Cram et al., Polyamine buffer for bivariate human flow cytogenetic analysis and sorting, Z. Darzynkiewicz, H. A. Crissman (Eds.), Methods in Cell Biology, Flow Cytometry, Vol. 33, Academic Press, San Diego, 1990, pp. 377-383. A volume of 2.5 μl of prewarmed chromosome 15 alpha-satellite centromeric probe fluorescein isothiocyanate (“FITC”) labeled (Cat. No. LPE015G; Cytocell, UK, distributor—Rainbow Scientific, Inc., Windsor, Conn., USA) was added to the chromosome suspension and the entire mixture was denatured for 3.5 minutes 73° C. Then, the suspension was kept on ice for about 5 minutes and incubated for about 2.5 hours at 37° C. in a shaking water bath. The labeled chromosome pellet was obtained by centrifugation and resuspension in 500 μl of prewarmed 0.1×SSC. Chromosomes were centrifuged and the pellet was resuspended in 2×SSC. An aliquot of 15 μl was stained with DAPI to make sure the labeling had taken place.

The chromosome 15-specific centromere probe is tagged with FITC fluorochromes. As the DNA probe that binds specifically to the target chromosome is fluorescently labeled with FITC, probe binding to the chromosome of interest is a type of FISH experiment. Even though the FISH technique is widely used on solid phases such as glass slides, FISH in suspension has been performed in limited fashion. See He et al., Fluorescence in situ hybridization of metaphase chromosomes in suspension, Int. J. Radiat. Biol. 77 (2001) 787-795. The previous problems encountered due to clump formation and loss of chromosomes were minimized in this recently developed procedure. Chromosome loss was decreased by fixing the chromosomes in methanol:acetic acid (3:1) as discussed above prior to exposing the chromosomes to the high temperatures of the FISH experiment and by omitting BSA and EDTA from the postwash buffer. Clump formation was also minimized first by eliminating the 10% dextran sulfate, which increases the viscosity of the hybridization buffer, and second, by decreasing the concentration of formamide from 70 to 40%. These revisions have resulted in preserving the morphology of the chromosomes and reducing sample loss.

The probe used to label chromosome 15 targets the alpha-satellite III region of the centromere was designated D15Z1. This probe was chosen because blocking of the centromeric region of the chromosome does not interfere with further analysis of the chromosomal DNA and histones, eliminating a complication associated with the removal of the hybridized probe if it binds to some other region of the chromosome. It has been reported that probes generated against this region can map to the short arm of chromosome 14. See Stergianou et al., High population incidence of the 15p marker D15Z1 mapping to the short arm of one homologue 14, Hum. Genet. 88 (1992) 364, 644; Stergianou, et al., A DA/DAPI positive human 14p heteromorphism defined by fluorescence in-situ hybridization using chromosome 15-specific probes D15Z1 (satellite III) and p-TRA-25 (alphoid), Hereditas 119 (1993) 105-110; Smeets et al., Chromosome 15p marker D15Z1 frequently maps to the short arm of other D-group chromosomes, Hum. Genet. 88 (1992) 365. Yet, those results were based on studies done on a very small sample population, and were seen on cell lines different from those used in the present application. Due to this controversy, this issue was addressed by characterizing the probe against metaphase spreads generated by GM01056C cells used in this study.

More specifically, to address this issue, the probe was characterized against metaphase spreads generated by the cell line used in this work. GM01056C cells on glass slides were used to perform solid-phase FISH experiments. Only two signals of equal intensity were seen, indicating binding of the probe to only one type of chromosome. This may mean that either that there is no cross-reactivity shown by this specific probe to this particular cell line used or, if there is any, it is undetectable under these conditions. The two instances previously reported involved different cell lines. In addition it is also noteworthy that the conditions for washing after hybridization were less stringent (2×SSC) than the one used in this procedure (0.1×SSC). In one of the reports, a nonfluorescence method of detection was employed, making the comparison of results even more difficult. To verify this point further, the centromere probe was reacted against a metaphase spread along with a chromosome 15-specific telomere probe, tetramethyl rhodamine isothiocyanate (“TRITC”) labeled (Cat. No. LPT 15QR; Cytocell, distributor—Rainbow Scientific, Inc.). As FIG. 2 illustrates, both probes show binding to the same two chromosomes in a spread, indicating the specificity of the centromere probe.

Linking Anti-FITC Antibody to Magnetic Beads

After the fluorescent-labeled probe is reacted with the chromosome pool, the target chromosomes are then identified by an antibody raised against FITC, which is covalently linked to a magnetic bead, enabling the bead-bound fraction to be separated from the remaining sample. A homobifunctional cross-linker, sulfo-EGS (ethylene glycol bis(sulfosuccinimidylsuccinate)) (Cat. No. 21566; Pierce, Rockford, Ill., USA), was used to link the anti-FITC antibody to the bead. The linker was chosen because it is cleavable by hydroxylamine so that the bead can be removed from the chromosome/probe conjugate after the separation process. Another important feature of the linker is its water solubility due to the sulfo group, making the handling procedure less complicated.

To prepare the anti-FITC antibody beads, equal amounts (8×10¹³ mol each) of rabbit anti-FITC antibody (Cat. No. 71-1900; Zymed Laboratories, South San Francisco, Calif., USA) and magnetic beads (Cat. No. BM546 nominal mean diameter of 1.63 microns, Bangs Laboratories, Inc., Fishers, Ind., USA) in PBS (pH 7.5) were reacted with a 10-fold molar excess of cross-linker, sulfo-EGS, dissolved in Nanopure water for about 30 minutes at room temperature. The reaction was quenched with a volume of 1 M Tris (pH 7.5) for about 15 minutes so that the final concentration of Tris was 20 to 50 mM. The reaction mixture was incubated for an additional 15 minutes. The beads were then collected using a magnet.

Bead Binding, Separation of Bead-Bound Fraction, and Cleavage of the Bead

Next, the beads containing the anti-FITC antibodies were contacted in suspension with the FITC-labeled chromosomes. More specifically, anti-FITC antibody-linked magnetic beads (1×10⁶) were suspended with 1×10⁶ probe-bound chromosomes in 100 μl of IB+M buffer containing 0.05% (v/v) Tween 20 and 5% (v/v) nonfat dry milk. The suspension was incubated for about 2 hours at 37° C. in a shaking water bath.

The suspension was then transferred to a square cuvette (so that separation could be performed on a flat surface) and the bead-bound fraction was collected by exposing one side of the cuvette to a bar magnet. The sample was then exposed to a magnetic field for about 30 to 40 minutes, and no settling of the particles was observed during this time during this time. The supernatant was removed and the cuvette was washed extensively with IB+M buffer prior to removing the magnetic field. Then, the bead-bound fraction was collected for further characterization.

When needed, the beads were cleaved by incubating equal volumes of sample and prewarmed 2.0 M hydroxylamine*HCl solution (pH 8.5) at 37° C. with stirring for 4 hours. The hydroxylamine*HCl solution was prepared by adding hydroxylamine*HCl to a phosphate buffer of pH 8.5 and then raising the pH to 8.5. The cleaved beads were collected by exposure to a magnetic field and the supernatant contained the cleaved chromosomes.

The magnetic bead-having the anti-FITC antibody may also be displaced from the target chromosome by reacting the bead-bound separated chromosome fraction with a 10,000 molar excess of free fluorescein dye molecules for 40 minutes at room temperature, thus displacing the chromosomes, and then collecting the supernatant containing free chromosomes upon exposure to a magnetic field, when required. Excess free fluorescein can be removed by centrifuging the sample at 350 g and aspirating the supernatant to collect the centrifuged pellet, which contains the chromosomes.

Paint FISH with Commercial Probe on Isolated Pool for Verification

To obtain a statistical output for the enrichment of the fractionated chromosomes in the isolated pool, a FISH experiment was performed with a commercially available probe specific for chromosome 15. The enrichment was indicated by the specific signal counted and compared to a nonspecific DNA binding signal. Thus, the specific probe was first characterized against a normal metaphase spread to confirm its specificity to chromosome 15 (the experimental procedure is similar to what is described under Paint FISH with commercial probe on isolated pool for verification). The control experiment for verification of enrichment by this procedure was performed with an aliquot of chromosomes immediately after extraction from cells, prior to isolation by the beads. A DNA-binding dye, DAPI, was used to identify the chromosomes on the slide. The slide was scanned to count the number of total chromosomes and painted chromosomes in different locations to obtain the ratio of painted/total, permitting calculation of the percentage enrichment.

More specifically, to determine if a homogeneous pool of chromosomes had been obtained, an aliquot of the isolated pool was characterized with a FISH probe that paints chromosome 15 specifically. A control experiment was performed with an aliquot prior to isolation. Experimental details are described below. The fractionated pool of chromosomes was spread on microscopic slides by two methods: cytocentrifuging 100 μl of the fractionated pool on a Shandon Cytospin (Thermo Electron Corp., Milford, Mass., USA) for about 10 minutes at 7.5 speed or about 5 μl smeared on a glass slide. In the case of cytocentrifugation, an absorbent filter card was placed between the funnel containing the cell suspension and the glass slide to absorb the excess liquid. The cytospun chromosomes were allowed to dry briefly in air and were then transferred for 5 minutes to a coplin jar containing water. Next, they were fixed in 3:1 methanol:acetic acid solution for 20 minutes at room temperature.

The paint FISH experiment was performed with a commercially available directly labeled whole-chromosome 15 paint probe (Cat. No. 33-120015, WCP 15q SpectrumGreen; Vysis, Inc., Downers Grove, Ill., USA), Ficoll Paque Plus (Cat. No. 17-1440-02; Amersham Biosciences, Piscataway, N.J., USA). Chromosome-containing slides were denatured for 5 minutes in the denaturation solution at 73+/−1° C. Then slides were dehydrated by immersing in a series of 70, 85, and 100% ethanol solutions for 1 minute each. Slides were then air dried and warmed to between 45 and 50° C. to evaporate any remaining ethanol. The probe mixture was prepared by mixing 1 μl of probe, 7 μl of hybridization buffer (provided with the probe kit), 2 μl of water to make the total volume 10 μl. The mixture was centrifuged for 1 to 3 seconds, vortexed, and then centrifuged again. The tube containing the probe mixture was placed in a 73 +/−1° C. water bath for 5 minutes. The tube was then removed from the water bath and placed at 45 to 50° C. until the probe was applied on the slide. When both slides and probe were ready, 10 μl of probe mixture was applied to target DNA, and a coverslip was placed on the specific area and sealed with nail polish. The slides were then incubated for 4 to 16 hours at 37° C. (preferably overnight) in a prewarmed humidified chamber for hybridization. Post-hybridization washes consist of two different washes: first, in 0.4×SSC at 73+/−1° C., agitating the slide-containing jar for 1 to 3 seconds and then leaving the slides in the jar for another 2 minutes; second, the slides were washed in 2×SSC at room temperature for a period of 5 to 60 seconds, agitating only for the first couple of seconds. Slides were allowed to air dry and counterstained with 10 ml of 0.125 μg/ml DAPI, prior to visualization under the appropriate filter set under a fluorescence microscope.

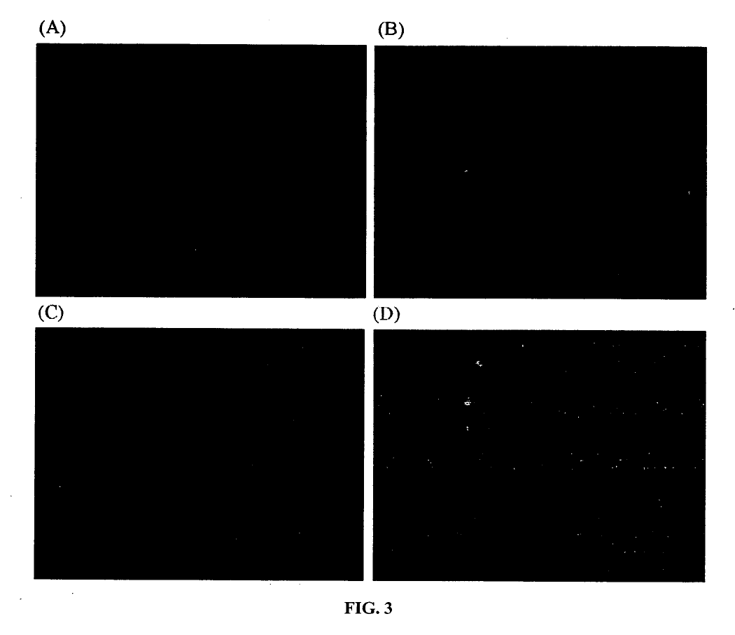

For the control experiment performed on an aliquot of extracted chromosomes prior to the fractionation process, the number obtained was about 6% (FIGS. 3A and 3B). Ideally, if the sample is random, this number should be around 4.35%, because there are two chromosomes 15 for every 46 chromosomes in a single cell. However, the slightly higher number observed is not unreasonable considering the fact that the chromosomes can come from more than one cell and do not segregate into groups from single cells as in a metaphase spread. When the same experiment was performed on the isolated fraction, a total of 239 chromosomes from three separate fractions were counted. From this set about 73% of the chromosomes identified by DAPI were painted, meaning that the enrichment by this fractionation procedure is approximately 73% (FIGS. 3C and 3D). When this same approach was adopted with a different probe specific for the subtelomeric region of chromosome 15 labeled with TRITC to verify the enrichment of fractionation, the percentage of enrichment was calculated to be about 80% with a total number of 110 chromosomes being counted. One of the reasons for this slight difference in enrichment could be due mainly to the difference in the accessibility of the two probes to the target DNA. Because the paint probe used in the first experiment covers the entire chromosome, it must hybridize to the entire chromosome as opposed to the second probe, which targets only the subtelomeric region in chromosome 15. The inability to enrich the pool 100% and obtain a homogeneous pool of chromosomes 15 could be attributed to any nonspecific binding of chromosomes to the magnetic beads and/or the centrifuge tubes during the process of separation. It is noteworthy that the integrity of histones was tested using anti-histone antibodies and it was concluded that they were still intact after this procedure. Although the extent of enrichment may be improved by multiple rounds (e.g., two or three rounds) of purification, sample loss could be a potential limitation. The extent of enrichment achieved by this method as the preliminary step of sample preparation is sufficient, however, as the specificity required can be achieved in the subsequent steps. One such possible approach is to design a probe that cleaves the specific genomic region that would lead to the extraction of histones in the region of interest. This type of separation of specific genomic intervals is more feasible with an enriched pool of the chromosome of interest, as demonstrated in this study.

Isolation of Genes and Associated Nucleosomes (Histones)

The overall objective of this work is to analyze and compare PTMs of histones in specific genomic loci in order to assess the differences in the modifications related to genetic diseases. To accomplish this task, histones need to be extracted from the particular region of the genome and analyzed. Clipping and isolation of the specific genomic region from the remainder is a prerequisite to extraction of the histones of interest. An enrichment of the chromosome on which the particular gene or genes of interest are located, makes it easier to isolate the specific region in the genome. The foregoing examples demonstrate a 73% enrichment of the chromosome of interest utilizing the magnetic bead approach. Even if the fractionated pool of chromosomes is not a 100% pure population, at the subsequent clipping to isolate the specific region, the anticipated purity can be obtained. Some possible approaches to clip the fragment of interest, in order to extract and analyze histones, are outlined below.

Restriction enzymes, also referred to as microscopic scalpels, can cleave double stranded DNA (ds-DNA) within a specific DNA sequence. Out of the different types of restriction enzymes, type II enzymes or endonucleases, which are phosphoesterases, hydrolyze the internucleotide bond of DNA or RNA molecules (see Mishra, N. C., John Wiley and Sons pg. 5 (2002)). The length of the DNA sequence identified by endonucleases, ranges from 4-8 base pairs. Two examples of such type II endonucleases are Sau3A1, is a 4-cutter enzyme identifying a 4 base long sequence, GATC, whereas EcoR1 identifies a 6 base long sequence of GAATTC (see Twyman, R. M., Advanced Molecular Biology; Bios Scientific, Oxford UK (1999)). The frequency of occurrence of a sequence recognized by these enzymes is dependent on the length of the recognition site (¼″ where n is the length of the site of recognition). The shorter the recognition site, the more frequent is its occurrence in a genome. Therefore, if a 4-cutter is used to digest the DNA, it generates fragments of 250 bp and a 6 cutter would generate a fragment 4000 bp in length. If one wishes to clip the entire PWS region, which is about 2 Mbp in length, from chromosome 15, these enzymes would not be the best choice. The closest one could get with endonucleases in performing such a cleavage in a complex, 3 billion base pair long genome such as the human genome, would be with an endonuclease that identifies a recognition site of 8 bases in length, which are referred to as a rare-cutter restriction enzyme. The probability of occurrence of an 8 base long sequence in the genome is about once every 65000 bases. Notl, although a rare-cutter enzyme, generates fragments of 95000 bp because its recognition site is fairly GC-rich and underrepresented in mammalian genomes (see Twyman (1999)).

An alternative approach is to cleave the genomic region of interest, which improves the specificity of cleavage because the target length of DNA is increased, using the synthetic nucleases. These are semi-synthetic site specific DNA cleavage reagents, composed of two chemical moieties. First component, a chelator and a suitable metal complex can cleave ds-DNA by Fenton chemistry as shown in below. A hydroxyl radical (.OH) is produced by the reaction of a chelator-metal complex such as Fe(II) EDTA complex or 1,10-phenanthroline-Cu(I) complex with H₂O₂, and cleaves the pentose sugar of the DNA backbone. Such metal chelator complexes are capable of cleaving double stranded DNA randomly, regardless of the sequence, is conjugated to the second component, a sequence specific DNA binding molecule that adds specificity to this reagent, thereby making the cleavage possible at a targeted specific DNA sequence. Such sequence specific DNA binding molecules include proteins (see Chen et al., Chemical Conversion of a DNA-Binding Protein into a Site-Specific Nuclease, Science 237, 1197-1201 (1987)), peptides (see Sluka et al., Synthesis of a Sequence-Specific DNA-Cleaving Peptide, Science 238, 1129-1132 (1987)), drugs (see Schultz et al., Design Synthesis of a Sequence-Specific DNA Cleaving Molecule (Distamycin-Edta)Iron(Ii), Journal of American Chemical Society 104, 6861-6863 (1982)), polyamide ligands (see Dervan et al., Sequence Specific DNA Recognition by Polyamides, Current opinion in chemical biology 3, 688-693 (1999)), and homopyrimidine oligonucleotides (see Moser et al., Sequence-Specific Cleavage of Double Helical DNA by Triple Helix Formation, Science 238, 645-648 (1987)) that can form triplexes. Escherichia coli Catabolite gene Activator Protein (CAP) is one such molecule that binds to DNA with extremely high affinity (4×10¹⁰ M⁻¹) and the DNA cleavage moiety is introduced at the helix-turn-helix motif of CAP. The main advantage of using such a reagent over a type II restriction endonuclease is the improved specificity due to the increase in length of the target DNA sequence which is 22 base pairs in this particular example (see Ebright et al., Conversion of a Helix-Turn-Helix Motif Sequence-Specific DNA Binding Protein into a Site-Specific DNA Cleavage Agent, Proceedings of the National Academy of Sciences of the United States of America 87, 2882-2886 (1990)) as opposed to 8 bases in restriction enzymes and data supports the specificity of the target sequence.
[Fe(EDT A)]²⁻+H₂O₂→[Fe(EDT A)]¹⁻+OH⁻+.OH
See Wolffe, A., Academic Press: Cambridge pp. 50-51 (1998))

Another possible approach along these same lines is to couple a random DNA cleavage reagent to a Zinc finger binding protein that binds a DNA sequence specifically. Such DNA binding zinc finger proteins can be conjugated through peptide linkers to increase the target DNA sequence (see Liu et al., Design of Polydactyl Zinc-Finger Proteins for Unique Addressing within Complex Genomes, Proceedings of the National Academy of Sciences of the United States of America 94, 5525-5530 (1997)). Therefore, depending on the sequence and the boundaries of the genomic region in focus, such exogenous DNA cleavage molecules can be designed. There are also some endogenous endonucleases such as 1-Tevl, a phage intron encoded endonuclease that is composed of a catalytic domain at the N-terminus and a C-terminus DNA binding domain with a long zinc finger linker that targets about 37 bases (see Dean et al., Zinc Finger as Distance Determinant in the Flexible Linker of Intron Endonuclease L-Tev, Proceedings of the National Academy of Sciences of the United States of America 99, 8554-8561 (2002)). Choosing the proper reagent or designing a new reagent will have to be determined by scanning the genomic sequence of interest and deciding the boundaries one would want to cleave the particular fragment, whether it be the entire PWS region or an individual gene in the PWS region.

A sucrose density gradient is one approach to separation of the clipped fragments from the remaining chromatin. The band expected for the clipped fragment in the sucrose gradient can be determined by its size and thus, isolated. Histones in the separated portion of chromatin can be extracted and further analyzed by mass spectrometry. Proteolytic digests of the extracted histones can be analyzed by either MALDI-MS/MS or ESI-MS/MS to study the PTMs (see Zhang, K., University of the Pacific (2000)). These approaches that utilize DNA cleaving reagents perform the isolation of the specific fragments in a parallel way, that consumes less time.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth in the figures are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A method for isolating and separating a target chromosome from a cellular sample comprising:
obtaining a cellular sample having a plurality of chromosomes, including the target chromosome;
arresting the cellular sample in metaphase;
extracting the plurality of chromosomes, including the target chromosome;
labeling the target chromosome with a nucleic acid probe having a fluorescent reporter group, said nucleic acid probe being specific for DNA of said target chromosome;
contacting said labeled target chromosome in suspension with an antibody against said fluorescent reporter group, said antibody covalently linked via a linker to magnetic bead, thereby forming a magnetically labeled target chromosome or segment thereof;
exposing said suspension to a magnetic field to separate said target chromosomes from said plurality of chromosomes.

2. The method of claim 1 wherein said target chromosome is human chromosome 15.

3. The method of claim 1 wherein said cellular sample contains only human chromosomes.

4. The method of claim 1 wherein magnetic particle is a ferric oxide particle.

5. The method of claim 1 wherein said magnetic particle has an average particle size between about 50 nm and 10 microns.

6. The method of claim 1 wherein said linker is ethylene glycol bis(sulfosuccinimidylsuccinate).

7. The method of claim 6 further comprising the step of cleaving said linker using hydroxylamine after said suspension is exposed to said magnetic field to separate said target chromosome from said magnetic particle.

8. The method of claim 1 wherein said fluorescent reporter group is fluorescein isothiocyanate.

9. The method of claim 1 wherein said extraction step comprises the step of lysing said cellular sample with a hypotonic solution to form a suspension of the plurality of chromosomes and other non-chromosomal cellular debris.

10. The method of claim 9 wherein said hypotonic solution further comprises a polyamine.

11. The method of claim 10 wherein said polyamine comprises spermine and spermidine.

12. The method of claim 9 wherein said non-chromosomal cellular debris is removed by centrifuging and washing with a polyamine chromosome isolation buffer comprising spermine and spermidine.

13. The method of claim 1 wherein said target chromosome is separated with a 73% or more purity.

14. The method of claim 1 wherein said target chromosome is separated with a 80% or more purity.

15. The method of claim 1 wherein said probe hybridizes a centromeric region of said target chromosome.

16. The method of claim 1 where said arresting the cellular sample in metaphase is performed by addition of colcemid to said sample.

17. The method of claim 1 wherein said arresting the cellular sample in metaphase is performed by the addition of colcemid to said sample, said extraction step comprises lysing said cellular sample with a hypotonic solution having a polyamine, said fluorescent reporter group is fluorescein isothiocyanate, and said linker is ethylene glycol bis(sulfosuccinimidylsuccinate).

18. The method of claim 1 wherein said fluorescent report group is fluorescein and further comprising the step of displacing said target chromosome from said magnetically labeled target chromosomes with excess fluorescein in suspension.

19. The method of claim 18 further comprising the step of exposing said suspension to a magnetic field, centrifuging the suspension, and then collecting a centrifuged pellet containing the target chromosome.

20. The method of claim 1 further comprising the step of isolating a gene from said target chromosome using restriction enzymes, synthetic or semi-synthetic nucleases, or a DNA cleavage agent coupled to a zinc finger binding protein.
